(19) United States
(12) Patent Application Publication
(10) Pub. No.: US 2014/0358229 A1
(43) Pub. Date: Dec. 4, 2014

(54) Medical Devices, Apparatuses, Systems, and Methods With Magnetic Shielding

(71) Applicant: Board of Regents, The University of Texas System, Austin, TX (US)

(72) Inventors: Richard A. BERGS, Grand Prairie, TX (US); Heather E. BEARDSLEY, Arlington, TX (US); Jeffrey A. CADEDDU, Dallas, TX (US); Raul FERNANDEZ, Arlington, TX (US); Daniel J. SCOTT, Dallas, TX (US)

(73) Assignee: Board of Regents, The University of Texas System, Austin, TX (US)

(21) Appl. No.: 14/244,493

(22) Filed: Apr. 3, 2014

Related U.S. Application Data
(63) Continuation of application No. 13/331,481, filed on Dec. 20, 2011, now abandoned.

(51) Int. Cl. A61B 19/00

(52) U.S. Cl. CPC A61B 19/22; A61B 2017/00876
USPC 623/11.11

(57) ABSTRACT

Embodiments of apparatuses, and methods and systems including apparatuses, configured to be magnetically coupled to a medical device within a body cavity of a patient. Some embodiments include one or more elements comprising at least one of a magnetically-attractive and magnetically-chargeable material; and a bumper extending around the one or more elements and configured to reduce the strength of a magnetic field of the one or more elements.

### CROSS-REFERENCE TO RELATED APPLICATION

This application is a continuation of U.S. patent application Ser. No. 13/331,481, filed Dec. 20, 2011, the disclosure of which is hereby incorporated herein in its entirety.

### BACKGROUND

1. Field of the Invention

The present invention relates generally to medical devices, apparatuses, systems, and methods, and, more particularly, but not by way of limitation, to medical devices, apparatuses, systems, and methods for performing medical procedures at least partially within a body cavity of a patient.

2. Description of Related Art

For illustration, the background is described with respect to medical procedures (e.g., surgical procedures), which can include laparoscopy, transmural surgery, and endoluminal surgery, including, for example, natural orifice transluminal endoscopic surgery (NOTES), single-incision laparoscopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. However, issues such as eye-hand dissociation, a two-dimensional field-of-view, instrumentation with limited degrees of freedom, and demanding dexterity requirements can pose challenges for many laparoscopic and endoscopic procedures. One limitation of laparoscopy can be the fixed working envelope surrounding each trocar. As a result, multiple ports may be used to accommodate changes in position of the instruments or laparoscope, for example, to improve visibility and efficiency. However, the placement of additional working ports may contribute to post-operative pain and increases risks, such as additional bleeding and adjacent organ damage.

The following published patent applications include information that may be useful in understanding the present medical devices, systems, and methods, and each is incorporated by reference in its entirety: (1) International Application No. PCT/US2009/063987, filed on Nov. 11, 2009, and published as WO 2010/056716; (2) U.S. patent application Ser. No. 10/024,636, filed Dec. 14, 2001, and published as Pub. No. US 2003/0114731; (3) U.S. patent application Ser. No. 10/999,396, filed Nov. 30, 2004, published as Pub. No. US 2005/0165449, and issued as U.S. Pat. No. 7,429,259; (4) U.S. patent application Ser. No. 11/741,731, filed Apr. 28, 2007, published as Pub. No. US 2007/0255273 and issued as U.S. Pat. No. 7,691,103; (5) U.S. patent application Ser. No. 12/146,953, filed Jun. 26, 2008, and published as Pub. No. US 2008/0269779; (6) International Patent Application No. PCT/US10/21292, filed Jan. 16, 2010, and published as WO 2010/083480.

### SUMMARY

This disclosure includes embodiments of apparatuses, systems, and methods.

Some embodiments of the present apparatuses comprise: a platform configured to be magnetically coupled to a medical device disposed within a body cavity of a patient through a tissue (e.g., where the platform comprises: one or more elements comprising at least one of a magnetically-attractive material and a magnetically-chargeable material); and a bumper extending around the one or more elements (e.g., where the bumper comprises: a magnetically-permeable material spaced apart from the one or more elements, the magnetically-permeable material configured to reduce the strength of a magnetic field of the one or more elements in at least one direction outside the bumper; and a magnetically-inert material surrounding at least a portion of the magnetically permeable material). In some embodiments, in at least one point one the bumper, a cross-sectional area of the non-magnetic material is greater than a cross-sectional area of the magnetically-permeable material. In some embodiments, a majority the bumper, the cross-sectional area of the non-magnetic material is greater than the cross-sectional area of the magnetically permeable bumper. In some embodiments, each of the one or more elements has a square cross-sectional shape. In some embodiments, each of the one or more elements comprises a magnet. In some embodiments, the bumper is spaced apart from an outer surface of the one or more elements by a substantially constant distance.

Some embodiments of the present apparatuses comprise: a platform configured to be magnetically coupled to a medical device disposed within a body cavity of a patient through a tissue (e.g., where the platform comprises: one or more elements comprising at least one of a magnetically-attractive material and a magnetically-chargeable material); and a bumper extending around the one or more elements (e.g., where the bumper comprises: a magnetically-permeable material spaced apart from the one or more elements, the magnetically-permeable material configured to reduce the strength of a magnetic field of the one or more elements in at least one direction outside the bumper). In some embodiments, the bumper further comprises a magnetically-inert material surrounding at least a portion of the magnetically-permeable material. In some embodiments, each of the one or more elements has a square cross-sectional shape. In some embodiments, each of the one or more elements comprises a magnet. In some embodiments, the bumper is spaced apart from an outer surface of the one or more elements by a substantially constant distance.

Some embodiments of the present apparatuses comprise: a platform configured to be magnetically coupled to a medical device disposed within a body cavity of a patient through a tissue (e.g., where the platform comprises: two or more elements each comprising at least one of a magnetically-attractive material and a magnetically-chargeable material); and a bumper extending around the two or more elements (e.g., where the bumper comprises: a magnetically-permeable material spaced apart from the two or more elements, the magnetically-permeable material configured to reduce the strength of a magnetic field of the one or more elements in at least one direction outside the bumper). In some embodiments, the bumper further comprises a magnetically-inert material surrounding at least a portion of the magnetically-permeable material. In some embodiments, each of the two or more elements has a square cross-sectional shape. In some embodiments, each of the two or more elements comprises a magnet. In some embodiments, the two or more elements comprises two elements having substantially opposite magnetic orientations. In some embodiments, the bumper is spaced apart from an outer surface of the two or more elements by a substantially constant distance. In some embodiments, the magnetically permeable material of the bumper has a substantially constant cross-sectional shape. In some embodiments, the cross-sectional shape is substantially rectangular. In some embodiments, the apparatus is disposed outside a body cavity of a patient and is magnetically coupled to a medical device within the body cavity.

Some embodiments of the present apparatuses comprise: a first platform configured to be inserted within a body cavity of a patient (e.g., where the first platform comprises: one or more elements comprising at least one of a magnetically-attractive material and a magnetically-chargeable material); and a second platform configured to be magnetically coupled to the first platform through a tissue (e.g., where the second platform comprises: one or more elements comprising at least one of a magnetically-attractive material and a magnetically-chargeable material; and a bumper extending around the one or more elements, the bumper comprising a magnetically-permeable material spaced apart from the one or more elements, the magnetically-permeable material configured to reduce the strength of a magnetic field of the one or more elements in at least one direction outside the bumper).

Any embodiment of any of the present apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 depicts a graphical representation of one of the present medical devices positioned within a body cavity of a patient and magnetically coupled to a positioning apparatus that is located outside the cavity.

FIG. 2 is an end view of the medical device and positioning apparatus shown in FIG. 1.

FIGS. 3A-3B depict a bottom view and a side cross-sectional view, respectively, respectively, of an embodiment of the positioning apparatus shown in FIG. 1.

FIG. 4 depicts a partially cutaway perspective view of one embodiment of the present apparatuses having a bumper.

FIG. 5 depicts a cross-sectional view of the bumper of FIG. 4.

FIGS. 6A-6B depict a second embodiment of the present apparatuses.

FIGS. 7A-7B depict a third embodiment of the present apparatuses.

FIGS. 8A-8B depict a fourth embodiment of the present apparatuses.

FIG. 9 depicts a top view of a portion of each of the apparatuses of FIGS. 6A-8B.

FIGS. 10A-10D depict the configuration and results of a first simulation performed for the apparatuses of FIGS. 6A-8B.

FIGS. 11A-13B depict the configurations and results of second and third simulations performed for the apparatuses of FIGS. 6A-8B.

FIGS. 14A-14C depict the configuration and results of a fourth simulation performed for the apparatuses of FIGS. 6A-8B.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a device or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a device or system that is configured in a certain way is configured in at least that way, but it can also be configured in other ways than those specifically described.

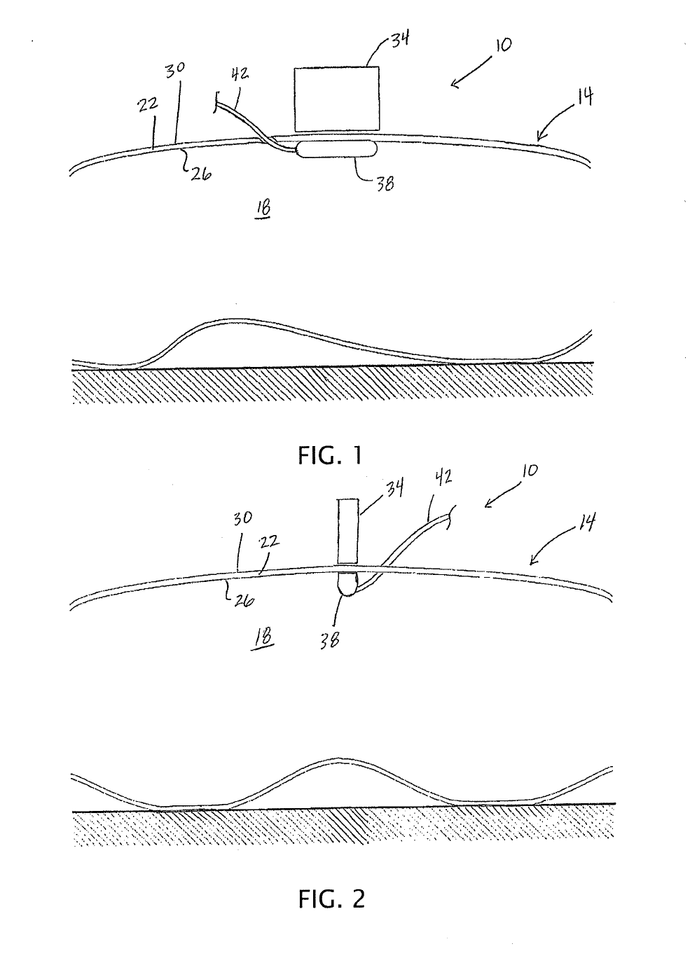

Referring now to the drawings, shown in FIGS. 1 and 2 by reference numeral 10 is one embodiment of a system for medical procedures that can be used with the present invention. System 10 is shown in conjunction with a patient 14, and more particularly in FIG. 1 is shown relative to a longitudinal cross-sectional view of the ventral cavity 18 of a human patient 14, and in FIG. 2 is shown relative to a transverse cross-sectional view of the ventral cavity of the patient. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIGS. 1 and 2, various embodiments of the present invention (including the version of system 10 shown in FIGS. 1 and 2) can also be used with other animals, such as in veterinary medical procedures.

Further, although system 10 is depicted relative to ventral cavity 18, system 10 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIGS. 1 and 2, system 10 comprises an apparatus 34 and a medical device 38; the apparatus is configured to magnetically position the device with a body cavity of a patient. In some embodiments, apparatus 34 can be described as an exterior apparatus and/or external unit and device 38 as an interior device and/or internal unit due the locations of their intended uses relative to patients. As shown, apparatus 34 can be positioned outside the cavity 18 near, adjacent to, and/or in contact with the exterior surface 30 of the patent 14. Device 38 is positionable (can be positioned), and is shown positioned, within the cavity 18 of the patient 14 and near, adjacent to, and/or in contact with the interior surface 26 of wall 22. Device 38 can be inserted or introduced into the cavity 18 in any suitable fashion. For example, the device 18 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with apparatus 34, such as by the methods described in this disclosure. If the cavity 18 is pressurized, device 38 can be inserted or introduced into the cavity 18 before or after the cavity 18 is pressurized.

Additionally, some embodiments of system 10 include a version of device 38 that has a tether 42 coupled to and extending away from the device 38. In the depicted embodiment, tether 42 extends from device 38 and out of the cavity 18, for example, through the opening (not shown) through which device 38 is introduced into the cavity 18. The tether 42 can be flexible and/or elongated. In some embodiments, the tether 42 can include one or more conduits for fluids that can be used, for example, for actuating a hydraulic cylinder or irrigating a region within the cavity 18. In some embodiments, the tether 42 can include one or more conductors for enabling electrical communication with the device 38. In some embodiments, the tether 42 can include one or more conduits for fluid and one or more conductors. In some embodiments, the tether does not include a conduit or conductor and, instead, includes a cord for positioning, moving, or removing device 38 from the cavity 18. The tether 14, for example, can be used to assist in positioning the device 34 while the device 34 is magnetically coupled to the apparatus 38, or to remove the device 34 from the cavity 18 when device 38 is not magnetically coupled to apparatus 34.

As is discussed in more detail below, apparatus 34 and device 38 can be configured to be magnetically couplable to one another such that device 38 can be positioned or moved within the cavity 18 by positioning or moving apparatus 34 outside the cavity 18. “Magnetically couplable” means capable of magnetically interacting so as to achieve a physical result without a direct physical connection. Examples of physical results are causing device 38 to move within the cavity 18 by moving apparatus 34 outside the cavity 18, and causing device 38 to remain in a position within the cavity 18 or in contact with the interior surface 26 of wall 22 by holding apparatus 34 in a corresponding position outside the cavity 18 or in contact with the exterior surface 30 of wall 22. Magnetic coupling can be achieved by configuring apparatus 34 and device 38 to cause a sufficient magnetic attractive force between them. For example, apparatus 34 can comprise one or more magnets (e.g., permanent magnets, electromagnets, or the like) and device 38 can comprise a ferromagnetic material. In some embodiments, apparatus 34 can comprise one or more magnets, and device 38 can comprise a ferromagnetic material, such that apparatus 34 attracts device 38 and device 38 is attracted to apparatus 34. In other embodiments, both apparatus 34 and device 38 can comprise one or more magnets such that apparatus 34 and device 38 attract each other.

The configuration of apparatus 34 and device 38 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when apparatus 34 and device 38 are magnetically coupled as shown, with each contacting a respective surface 26 or 30 of wall 22, the magnetic force between them can compress wall 22 to some degree such that wall 22 exerts a spring or expansive force against apparatus 34 and device 38, and such that any movement of apparatus 34 and device 38 requires an adjacent portion of wall 22 to be similarly compressed. Apparatus 34 and device 38 can be configured to overcome such an impeding force to the movement of device 38 with apparatus 34. Another force that the magnetic attractive force between the two may have to overcome is any friction that exists between either and the surface, if any, that it contacts during a procedure (such as apparatus 34 contacting a patient's skin). Another force that the magnetic attractive force between the two may have to overcome is the force associated with the weight and/or tension of the tether 42 and/or frictional forces on the tether 42 that may resist, impede, or affect movement or positioning of device 38 using apparatus 34.

In some embodiments, device 38 can be inserted into cavity 18 through an access port having a suitable internal diameter. Such access ports includes those created using a conventional laparoscopic trocar, gel ports, those created by incision (e.g., abdominal incision), and natural orifices. Device 38 can be pushed through the access port with any elongated instrument such as, for example, a surgical instrument such as a laparoscopic grasper or a flexible endoscope.

In embodiments where the tether 42 is connectable to a power source or a hydraulic source (not shown), the tether can be connected to the power source or the hydraulic source (which may also be described as a fluid source) either before or after it is connected to device 38.

In some embodiments, when device 38 is disposed within cavity 18, device 38 can be magnetically coupled to apparatus 34. This can serve several purposes including, for example, to permit a user to move device 38 within cavity 18 by moving apparatus 34 outside cavity 18. The magnetic coupling between the two can be affected by a number of factors, including the distance between them. For example, the magnetic attractive force between device 38 and apparatus 34 increases as the distance between them decreases. As a result, in some embodiments, the magnetic coupling can be facilitated by temporarily compressing the tissue (e.g., the abdominal wall) separating them. For example, after device 38 has been inserted into cavity 18, a user (such as a surgeon) can push down on apparatus 34 (and wall 22) and into cavity 18 until apparatus 34 and device 38 magnetically couple.

In FIGS. 1 and 2, apparatus 34 and device 38 are shown at a coupling distance from one another and magnetically coupled to one another such that device 38 can be moved within the cavity 18 by moving apparatus 34 outside the outside wall 22. The “coupling distance” between two structures (e.g., apparatus 34 and device 38) is defined as a distance between the closest portions of the structures at which the magnetic attractive force between them is great enough to permit them to function as desired for a given application.

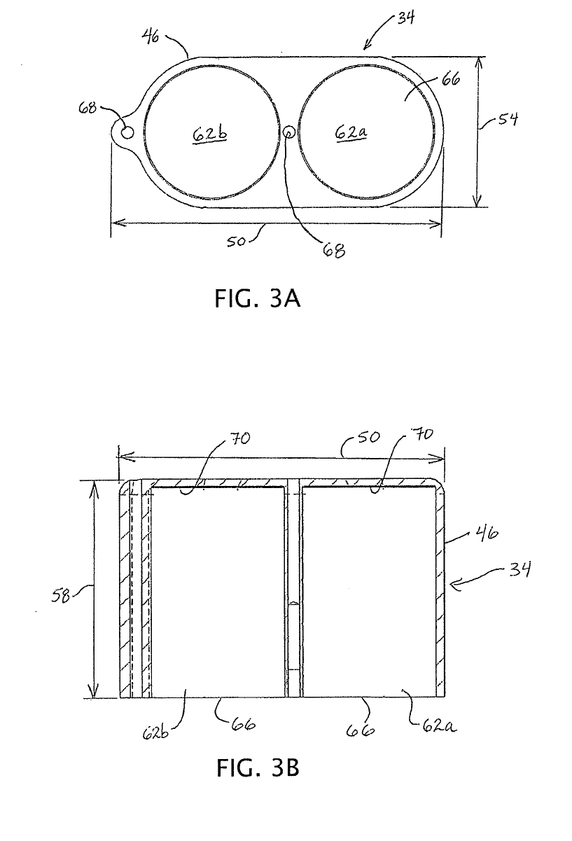

Referring now to FIGS. 3A and 3B, a bottom view and a side cross-sectional view are shown, respectively, of an embodiment of apparatus 34. Apparatus 34 has a width 50, a depth 54, and a height 58, and includes a housing 46. The apparatus (and, more specifically, housing 46) is configured to support, directly or indirectly, at least one magnetic assembly in the form of one or more magnetic field sources. In the embodiments shown, apparatus 34 is shown as including a first magnetic field source 62a and a second magnetic field source 62b. Each magnetic field source 62a, 62b has a coupling end 66 and a distal end 70. As described in more detail below, the coupling ends face device 38 when apparatus 34 and device 38 are magnetically coupled. The depicted embodiment of housing 46 of apparatus 34 also includes a pair of guide holes 68 extending through housing 46 for guiding, holding, or supporting various other devices or apparatuses, as described in more detail below. In other embodiments, the housing of apparatus 34 can have any other suitable number of guide holes 68 such as, for example, zero, one, three, four, five, or more guide holes 68. In some embodiments, housing 46 comprises a material that is minimally reactive to a magnetic field such as, for example, plastic, polymer, fiberglass, or the like. In other embodiments, housing 46 can be omitted or can be integral with the magnetic field sources such that the apparatus is, itself, a magnetic assembly comprising a magnetic field source.

Magnets, in general, have a north pole (the N pole) and a south pole (the S pole). In some embodiments, apparatus 34 can be configured (and, more specifically, its magnetic field sources can be configured) such that the coupling end 66 of each magnetic field source is the N pole and the distal end 70 of each magnetic field source is the S pole. In other embodiments, the magnetic field sources can be configured such that the coupling end 66 of each magnetic field source is the S pole and the distal end 70 of each magnetic field source is the N pole. In other embodiments, the magnetic field sources can be configured such that the coupling end of the first magnetic field source 62a is the N pole and the recessed end of the first magnetic field source 62a is the S pole, and the coupling end of the second magnetic field source 62b is the S pole and the recessed end of the second magnetic field source 62b is the N pole. In other embodiments, the magnetic field sources can be configured such that the coupling end of the first magnetic field source 62a is the S pole and its recessed end is the N pole, and the coupling end of the second magnetic field source 62b is the N pole and its recessed end is the S pole.

In the embodiment shown, each magnetic field source includes a solid cylindrical magnet having a circular cross section. In other embodiments, each magnetic field source can have any suitable cross-sectional shape such as, for example, rectangular, square, triangular, fanciful, or the like. In some embodiments, each magnetic field source comprises any of: any suitable number of magnets such as, for example, one, two, three, four, five, six, seven, eight, nine, ten, or more magnets; any suitable number of electromagnets such as, for example, one, two, three, four, five, six, seven, eight, nine, ten or more electromagnets; any suitable number of pieces of ferromagnetic material such as, for example, one, two, three, four, five, six, seven, eight, nine, ten or more pieces of ferromagnetic material; any suitable number of pieces of paramagnetic material such as, for example, one, two, three, four, five, six, seven, eight, nine, ten or more pieces of paramagnetic material; or any suitable combination of magnets, electromagnets, pieces of ferromagnetic material, and/or pieces of paramagnetic material.

In some embodiments, each magnetic field source can include four cylindrical magnets (not shown) positioned in end-to-end in linear relation to one another, with each magnet having a height of about 0.5 inch and a circular cross-section that has a diameter of about 1 inch. In these embodiments, the magnets can be arranged such that the N pole of each magnet faces the S pole of the next adjacent magnet such that the magnets are attracted to one another and not repulsed.

In some embodiments, device 38 can also include one or more magnets or other magnetically-attractive elements that can be attracted to magnetic field sources 62a and 62b to enable magnetic coupling between apparatus 34 and 38.

Examples of suitable magnets can include: flexible magnets; Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929. In some embodiments, one or more magnetic field sources can comprise ferrous materials (e.g., steel) and/or paramagnetic materials (e.g., aluminum, manganese, platinum).

In some embodiments, apparatus 34 and device 38 can be configured to have a minimum magnetic attractive force or “coupling force” at a certain distance. For example, in some embodiments, apparatus 34 and device 38 can be configured such that at a distance of 50 millimeters between the closest portions of apparatus 34 and device 38, the magnetic attractive force between apparatus 34 and device 38 is at least about: 20 grams, 25 grams, 30 grams, 35 grams, 40 grams, or 45 grams. In some embodiments, apparatus 34 and device 38 can be configured such that at a distance of about 30 millimeters between the closest portions of apparatus 34 and device 38, the magnetic attractive force between them is at least about: 25 grams, 30 grams, 35 grams, 40 grams, 45 grams, 50 grams, 55 grams, 60 grams, 65 grams, 70 grams, 80 grams, 90 grams, 100 grams, 120 grams, 140 grams, 160 grams, 180 grams, or 200 grams. In some embodiments, apparatus 34 and device 38 can be configured such that at a distance of about 15 millimeters between the closest portions of apparatus 34 and device 38, the magnetic attractive force between them is at least about: 200 grams, 250 grams, 300 grams, 350 grams, 400 grams, 45 grams, 500 grams, 550 grams, 600 grams, 650 grams, 700 grams, 800 grams, 900 grams, or 1000 grams. In some embodiments, apparatus 34 and device 38 can be configured such that at a distance of about 10 millimeters between the closest portions of apparatus 34 and device 38, the magnetic attractive force between them is at least about: 500 grams, 1000 grams, 2000 grams, 2200 grams, 2400 grams, 2600 grams, 2800 grams, 3000 grams, 3200 grams, 3400 grams, 3600 grams, 3800 grams, or 4000 grams.

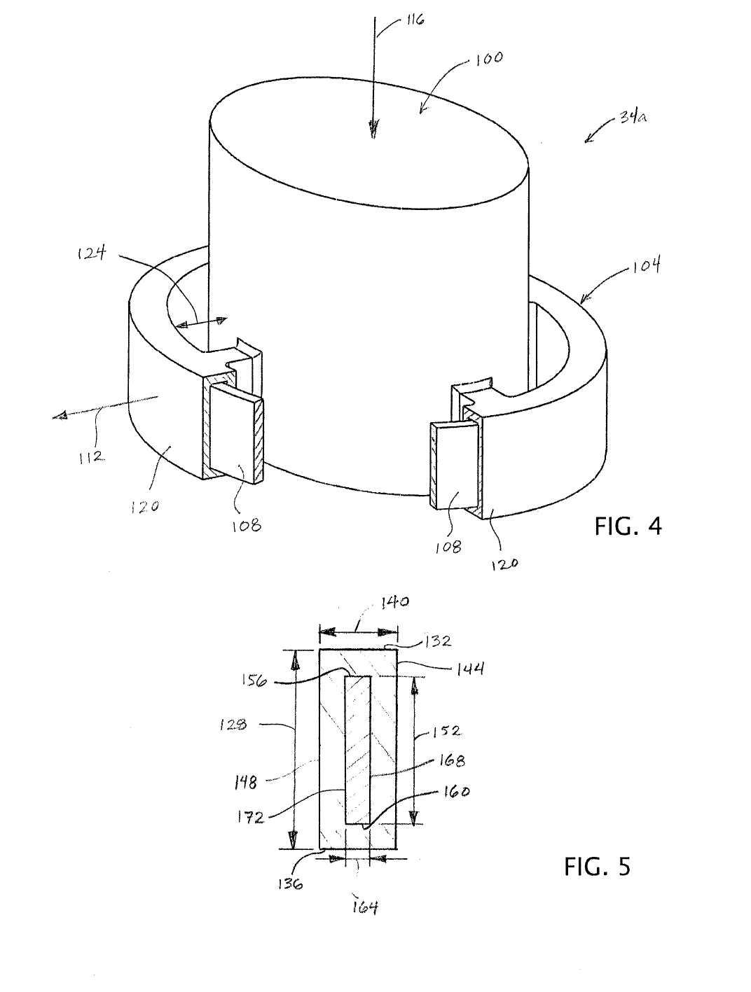

FIG. 4 depicts a partially-cutaway perspective view of one embodiment 34a of the present apparatuses that is configured to be magnetically coupled to a medical device (e.g., 38) disposed within a body cavity of a patient through a tissue. In the embodiment shown, apparatus 34a comprises a platform 100 that includes one or more (e.g., two or more) elements comprising at least one of a magnetically-attractive material and a magnetically-chargeable material (not specifically shown in FIG. 4, but such as, for example, similar to magnetic field sources 62a and 62b, described above); and a bumper 104 and extending around the one or more elements. In the embodiment shown, bumper 104 comprises: a magnetically-permeable material 108 configured to reduce the strength of a magnetic field of the one or more elements in at least one direction outside the bumper (e.g., such as direction 112 that is laterally outward relative to the one or more elements and/or perpendicular to direction 116 in which the one or more elements are magnetized and/or magnetizable). Such a reduction in the strength of the magnetic field (e.g., at a point a certain distance from the one or more elements) can be advantageous in reducing the attraction of objects (e.g., scalpels, forceps, etc.) that include ferromagnetic and/or paramagnetic material, and/or reducing the distance required between adjacent apparatuses 34a at which magnetic interactions between the adjacent apparatuses are manageable (e.g., do not significantly interfere with a user's ability to move the apparatuses relative to one another or the apparatuses interactions with respective medical devices 38).

In the embodiment shown, bumper 104 also comprises a magnetically-inert material 120 surrounding at least a portion of magnetically permeable material 108. Examples of magnetically-permeable materials include a ferromagnetic materials (e.g., iron, steel, etc.) and paramagnetic materials (e.g., platinum). Examples of magnetically-inert materials include various plastics, polymers, and the like. In the embodiment shown, bumper 104 is configured such that magnetically-permeable material 108 is spaced apart from the one or more elements by a distance 124. Distance 124 can, for example, be equal to, greater than, or between any of: 0.25, 0.5, 0.75, 1.0, 1.5, or more inches. In the embodiment shown, the one or more elements can extend between a first or coupling end 66 and a second or distal end 70, and bumper 104 is disposed at or near coupling end 66. In other embodiments, bumper 104 can be disposed at or near distal end 70, or at any suitable point between coupling end 66 and distal end 70. For example, bumper 104 can be centered at the midpoint between coupling end 66 and distal end 70.

FIG. 5 depicts a cross-sectional view of bumper 104. In the embodiment shown, bumper 104 has a rectangular cross-sectional shape in which magnetically-permeable material 108 and magnetically-inert material 120 each has a cross-sectional shape. In this embodiment, bumper 104 has a height 128 extending between a top 132 and a bottom 136, and has a width 140 extending between an inner side 144 and an outer side 148. Height 128 can, for example, be equal to, greater than, or between any of: 0.25, 0.5, 0.75, 1.0, 1.5, or more inches. Width 140 can, for example, be equal to, greater than, or between any of: 0.1, 0.2, 0.25, 0.375, 0.5, or more inches. Similarly, in the embodiment shown, magnetically permeable material 108 has a height 152 extending between a top 156 and a bottom 160, and has a width 164 extending between an inner side 168 and an outer side 172. Height 152 can, for example, be equal to, greater than, or between any of: 0.25, 0.5, 0.75, 1.0, 1.5, or more inches. Width 164 can, for example, be equal to, greater than, or between any of: 0.05, 0.1, 0.25, 0.5, or more inches.

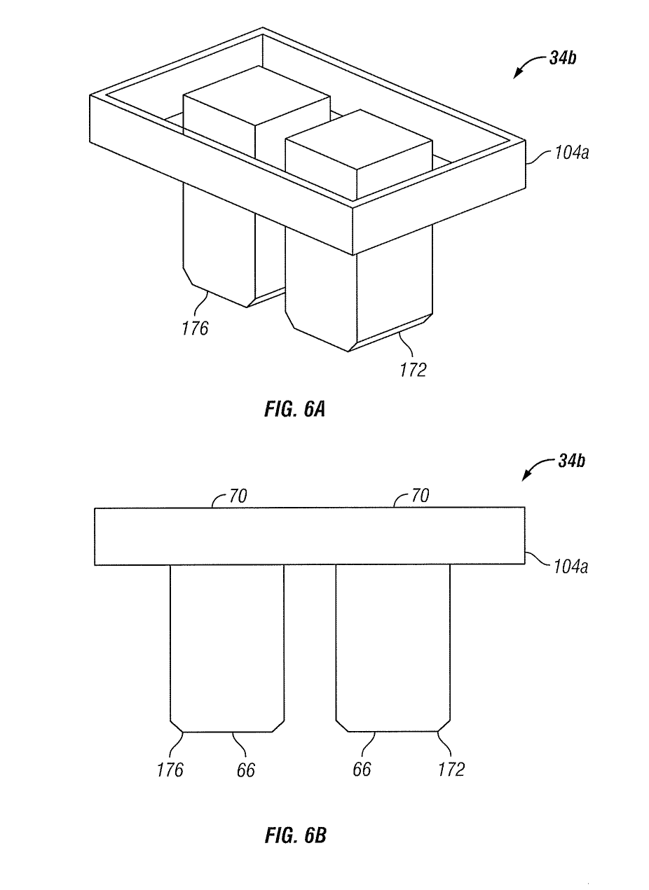

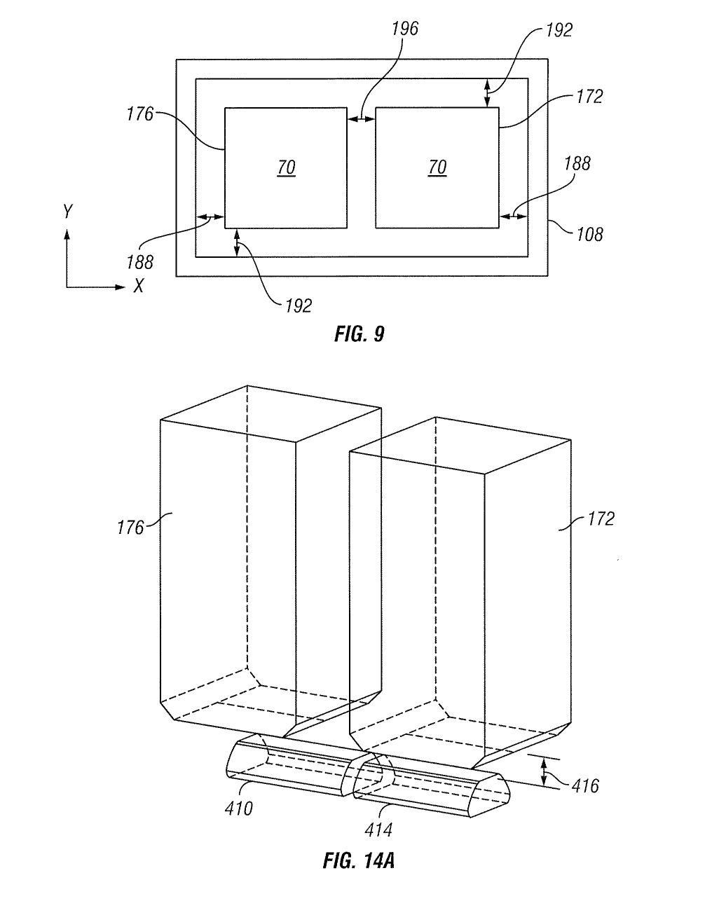

FIGS. 6A-6B depict a second embodiment 34b of the present apparatuses. Apparatus 34b is substantially similar in some respects to apparatus 34a. As such, the differences between apparatus 34b and apparatus 34a are primarily described here. In the embodiment shown, apparatus 34b comprises a platform 168 with two elements (first element 172 and second element 176) each comprising at least one of a magnetically-attractive material and a magnetically-chargeable material. First and second elements 172 and 176 can, for example, be similar to magnetic field sources 62a and 62b, described above, with the primary exception that first and second elements 172 and 176 each have the shape of an elongated cylinder with a square cross-sectional shape. Apparatus 34b also comprises a bumper 104a extending around the two elements. Bumper 104a is similar to bumper 104, with the primary exception that bumper 104a has a rectangular shape when viewed from the top (FIG. 9), rather than the oval shape of bumper 104. In the embodiment shown, bumper 104a is at a bottom position at or near coupling ends 66 of first and second elements 172 and 176 (e.g., such that bottom 136 of bumper 104 or bottom 160 of magnetically-permeable material 108 is substantially even with the coupling ends of the first and second elements.

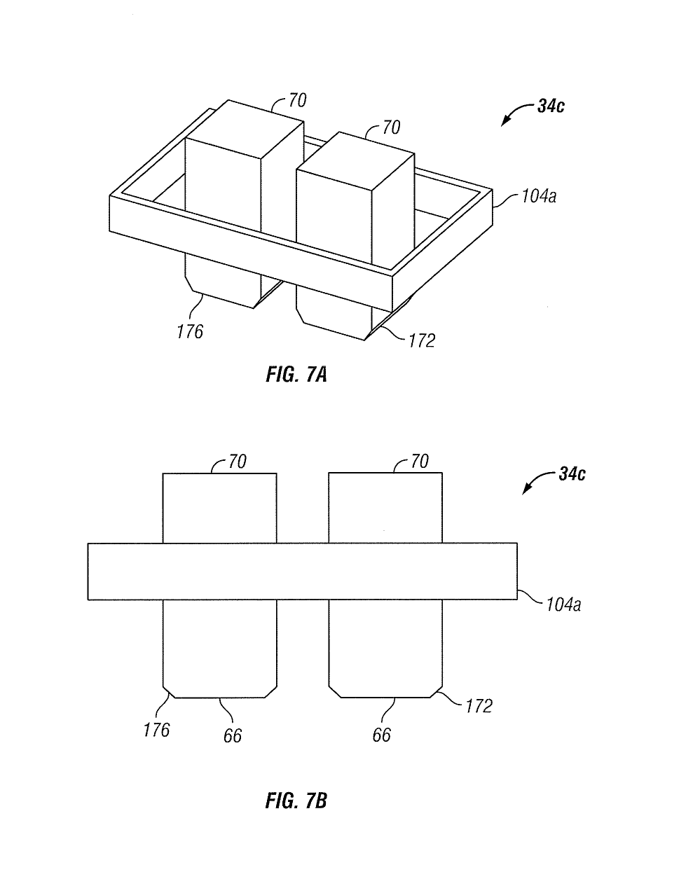

FIGS. 7A-7B depict a third embodiment 34c of the present apparatuses. Apparatus 34c is substantially similar to apparatus 34b, with the exception that bumper 104a is at a middle position centered at the midpoint between coupling ends 66 and distal ends 70 of first and second elements 172 and 176, such that a distance 180 between coupling ends 66 and bottom 136 of bumper 104a is substantially equal to a distance 184 between distal ends 70 and top 132. In other embodiments, distance 180 can be any suitable size, such as, for example, equal to, between, or greater than any of: 10%, 20%, 30%, 40%, 50% or more of the overall distance between coupling end 66 and distal end 70 of either of first and second elements 172 and 176.

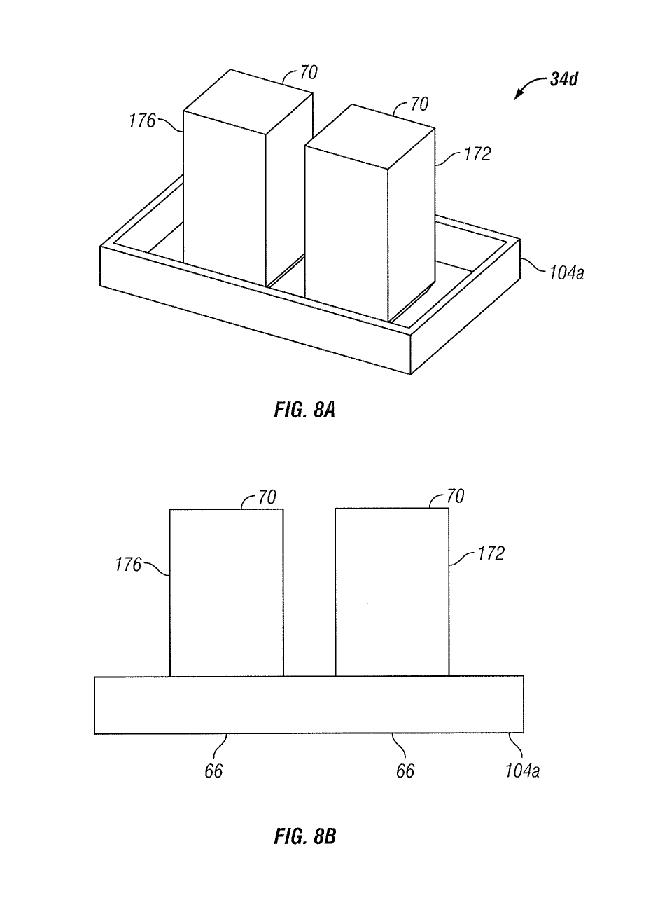

FIGS. 8A-8B depict a fourth embodiment 34d of the present apparatuses. Apparatus 34d is substantially similar to apparatus 34b, with the exception that bumper 104a is at a top position at or near distal ends 70 of first and second elements 172 and 176 (e.g., such that top 132 of bumper 104 or top 156 of magnetically-permeable material 108 is substantially even with the distal ends of the first and second elements.

FIG. 9 depicts a top plan view of a portion of any of apparatuses 34b, 34c, and 34d (all appear identical in this view) showing the relation between magnetically-permeable material 108 of the bumper and first and second elements 172 and 176. In the embodiment shown, magnetically-permeable material 108 is spaced apart from first and second elements 172 and 176 in an X-direction by a distance 188, and in a Y-direction by a distance 192. Distances 188 and 192 can, for example, be equal to, greater than, or between any of: 0.1, 0.25, 0.5, 0.75, 1.0, or more inches. In the embodiment shown, first element 172 and first element 176 each has a square shape viewed from the top, and are spaced apart from each other by distance 196. Distance 196 can, for example, be equal to, greater than, or between any of: 0.1, 0.25, 0.5, 0.75, 1.0, or more inches. In the embodiment shown, distance 196 is substantially equal to distances 188 and 192.

Various computer simulations were performed for apparatuses 34b, 34c, and 34d, and compared to an apparatus without bumper 104a, to approximate the effects of the present bumpers on elements (172 and 176) comprising magnets. In each such simulation, the bumper of FIG. 9 with the cross-section of FIG. 5 was modeled in two configurations with two different magnetically-permeable materials, and at each of the three locations (bottom, middle, and top) depicted in FIGS. 6A-8A. The first configuration of bumper 104a, referred to in this disclosure as Shield-1 included a magnetically-permeable material 108 having a width 152 of 3.175 millimeters (mm) or 0.125 inches (in.), and a height 164 of 12.7 mm or 0.5 in., spaced apart from first and second elements 172 and 176 by distances 196 and 200 of 0.5 mm or 0.02 in. The first configuration was simulated with two different materials: AISI 1010 steel and Carpenter 49 steel. The second configuration of bumper 104b, referred to in this disclosure as Shield-2, included a magnetically-permeable material 108 having a width 152 of 3.175 millimeters (mm) or 0.125 inches (in.), and a height 164 of 12.7 mm or 0.5 in., spaced apart from first and second elements 172 and 176 by distances 196 and 200 of 12.7 mm or 0.5 in. The second configuration was tested with only AISI 1010 steel.

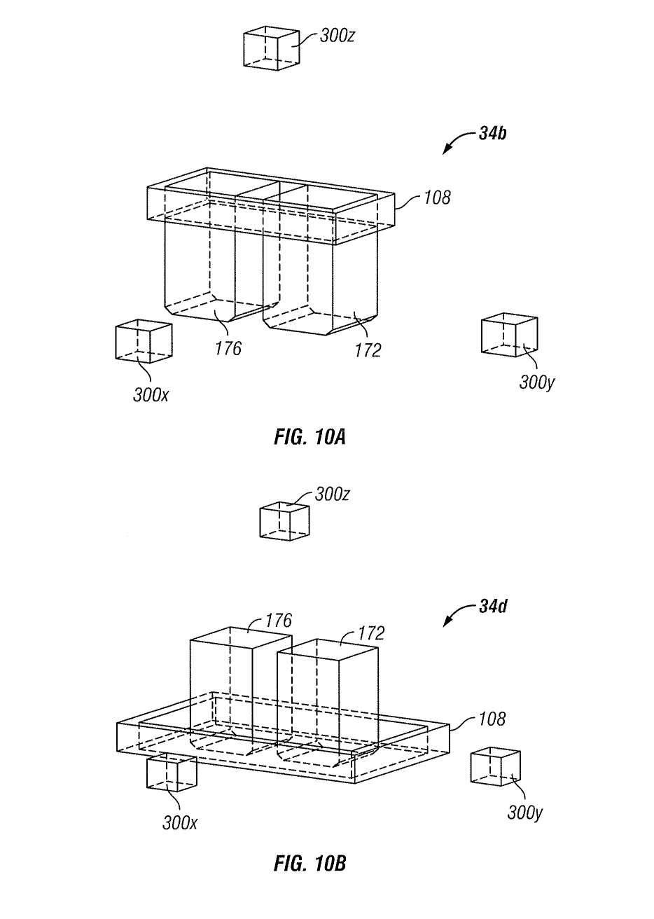

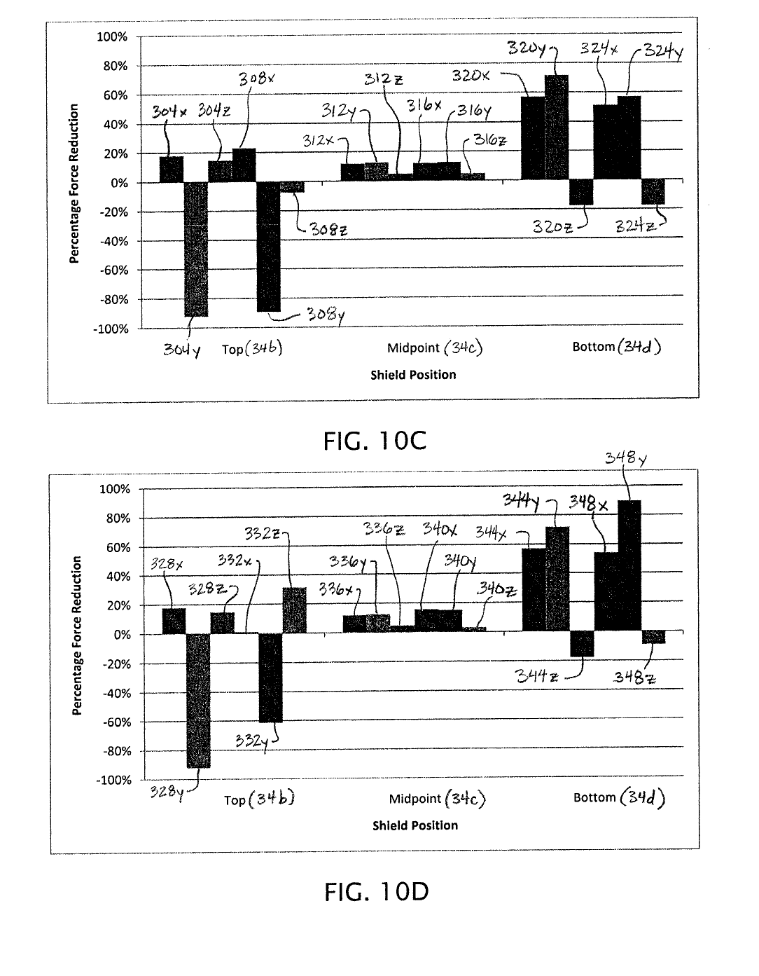

FIGS. 10A-10B depict perspective views of apparatuses 34b and 34d, respectively, in the configuration of a first simulation. For illustration, FIG. 10A depicts apparatus 34b with the Shield-1 dimensions (relatively smaller gap or space between magnetically-permeable material 108 and first and second elements 172 and 176), and FIG. 10B depicts apparatus 34d with the Shield-2 dimensions (relatively larger gap or space between magnetically-permeable material 108 and first and second elements 172 and 176). Apparatus 34c was also simulated in this configuration. In the configuration shown, the apparatuses were simulated with 12.7 mm or 0.5 in. cubes 300x, 300y, and 300z spaced 50 mm or 2 in. in X, Y, and Z directions, respectively, from first and/or second element 172 and/or 176. The cubes are representative of clamps, scalpels, or items that may be found in surgical fields. The magnitude of the magnetic force on the respective cubes was compared to the magnetic force on the respective cubes generated by the first and second elements without the bumper.

FIGS. 10C-10D depict the results of the simulations of FIGS. 10A-10B. FIG. 10C depicts the reduction in force felt by each block 300 in the X-direction, Y-direction, and Z-direction, respectively, for the apparatuses 34b, 34c, and 34d in which magnetically-permeable material 108 with the Shield-1 dimensions comprises either AISI 1010 steel or Carpenter 49 steel, relative to a similar apparatus without a bumper 104a. As such, a positive percentage force reduction in the chart corresponds to a reduction in force felt by the corresponding block. Bars 304x, 304y, and 304z correspond to the percentage reduction in force felt by blocks 300x, 300y, and 300z for apparatus 34b in which magnetically-permeable material 108 comprises AISI 1010 steel. Bars 308x, 308y, and 308z correspond to the percentage reduction in force felt by blocks 300x, 300y, and 300z for apparatus 34b in which magnetically-permeable material 108 comprises Carpenter 49 steel. Bars 312x, 312y, and 312z correspond to the percentage reduction in force felt by blocks 300x, 300y, and 300z for apparatus 34c in which magnetically-permeable material 108 comprises AISI 1010 steel. Bars 316x, 316y, and 316z correspond to the percentage reduction in force felt by blocks 300x, 300y, and 300z for apparatus 34c in which magnetically-permeable material 108 comprises Carpenter 49 steel. Bars 320x, 320y, and 320z correspond to the percentage reduction in force felt by blocks 300x, 300y, and 300z for apparatus 34c in which magnetically-permeable material 108 comprises AISI 1010 steel. Bars 324x, 324y, and 324z correspond to the percentage reduction in force felt by blocks 300x, 300y, and 300z for apparatus 34c in which magnetically-permeable material 108 comprised Carpenter 49 steel.

FIG. 10D depicts the reduction in force felt by each block 300 in the X-direction, Y-direction, and Z-direction, respectively, for apparatus 34c in which magnetically-permeable material 108 with either the Shield-1 or Shield-2 dimensions comprises AISI 1010 steel, relative to a similar apparatus without a bumper 104a. Bars 328x, 328y, and 328z correspond to the percentage reduction in force felt by blocks 300x, 300y, and 300z for apparatus 34c in which magnetically-permeable material 108 has the Shield-1 dimensions. Bars 332x, 332y, and 332z correspond to the percentage reduction in force felt by blocks 300x, 300y, and 300z for apparatus 34c in which magnetically-permeable material 108 has the Shield-2 dimensions. Bars 336x, 336y, and 336z correspond to the percentage reduction in force felt by blocks 300x, 300y, and 300z for apparatus 34c in which magnetically-permeable material 108 has the Shield-1 dimensions. Bars 340x, 340y, and 340z correspond to the percentage reduction in force felt by blocks 300x, 300y, and 300z for apparatus 34c in which magnetically-permeable material 108 has the Shield-2 dimensions. Bars 344x, 344y, and 344z correspond to the percentage reduction in force felt by blocks 300x, 300y, and 300z for apparatus 34c in which magnetically-permeable material 108 has Shield-1 dimensions. Bars 348x, 348y, and 348z correspond to the percentage reduction in force felt by blocks 300x, 300y, and 300z for apparatus 34c in which magnetically-permeable material 108 has the Shield-2 dimensions.

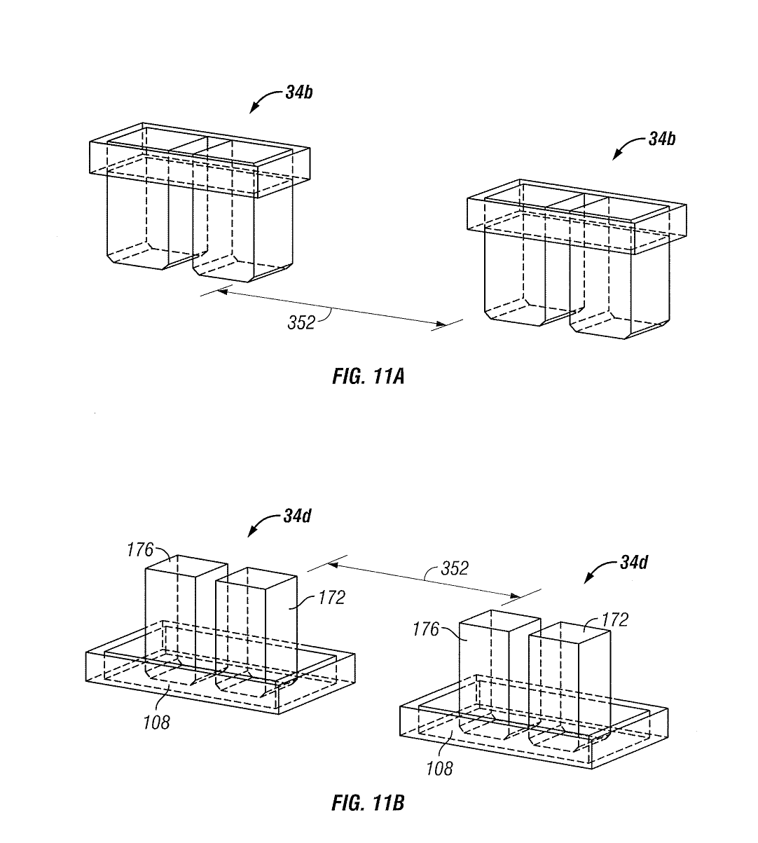

FIGS. 11A-11B depict perspective views of apparatuses 34b and 34d, respectively, in the configuration of a second simulation. More particularly, in the embodiment shown, two apparatuses 34b or 34d are disposed next to each other at a distance 352 in an X-direction between their respective first and second elements 172 and 176. In the simulations performed, distance 352 was 100 mm or 4 in. For illustration, FIG. 11A depicts apparatuses 34b with the Shield-1 dimensions (relatively smaller gap or space between magnetically-permeable material 108 and first and second elements 172 and 176), and FIG. 11B depicts apparatuses 34d with the Shield-2 dimensions (relatively larger gap or space between magnetically-permeable material 108 and first and second elements 172 and 176). Apparatus 34c was also simulated in this configuration.

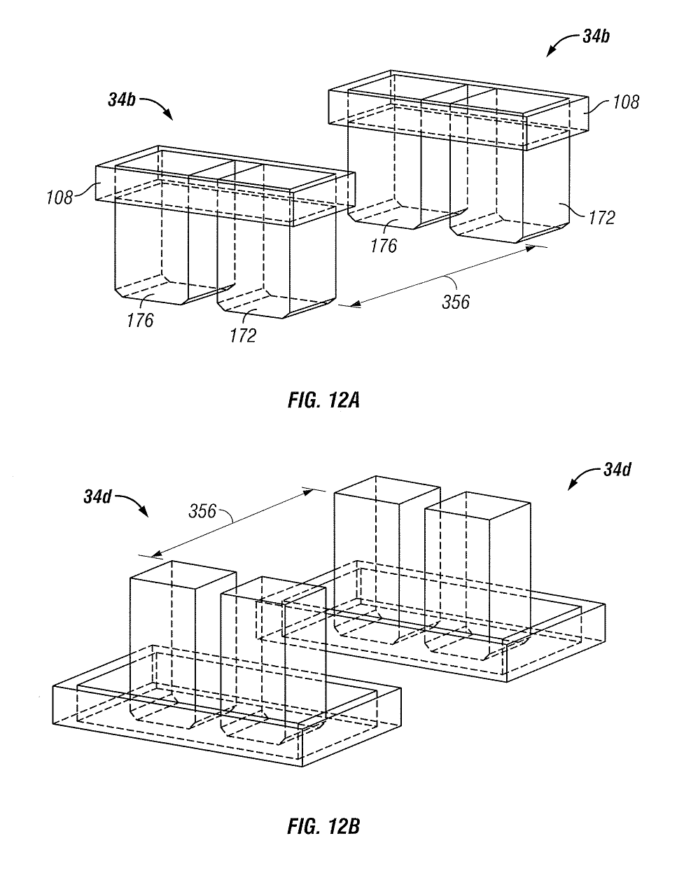

FIGS. 12A-12B depict perspective views of apparatuses 34b and 34d, respectively, in the configuration of a third simulation. More particularly, in the embodiment shown, two apparatuses 34b or 34d are disposed next to each other at a distance 356 in an Y-direction between their respective first and second elements 172 and 176. In the simulations performed, distance 356 was 100 mm or 4 in. For illustration, FIG. 12A depicts apparatuses 34b with the Shield-1 dimensions (relatively smaller gap or space between magnetically-permeable material 108 and first and second elements 172 and 176), and FIG. 12B depicts apparatuses 34d with the Shield-2 dimensions (relatively larger gap or space between magnetically-permeable material 108 and first and second elements 172 and 176). Apparatus 34c was also simulated in this configuration.

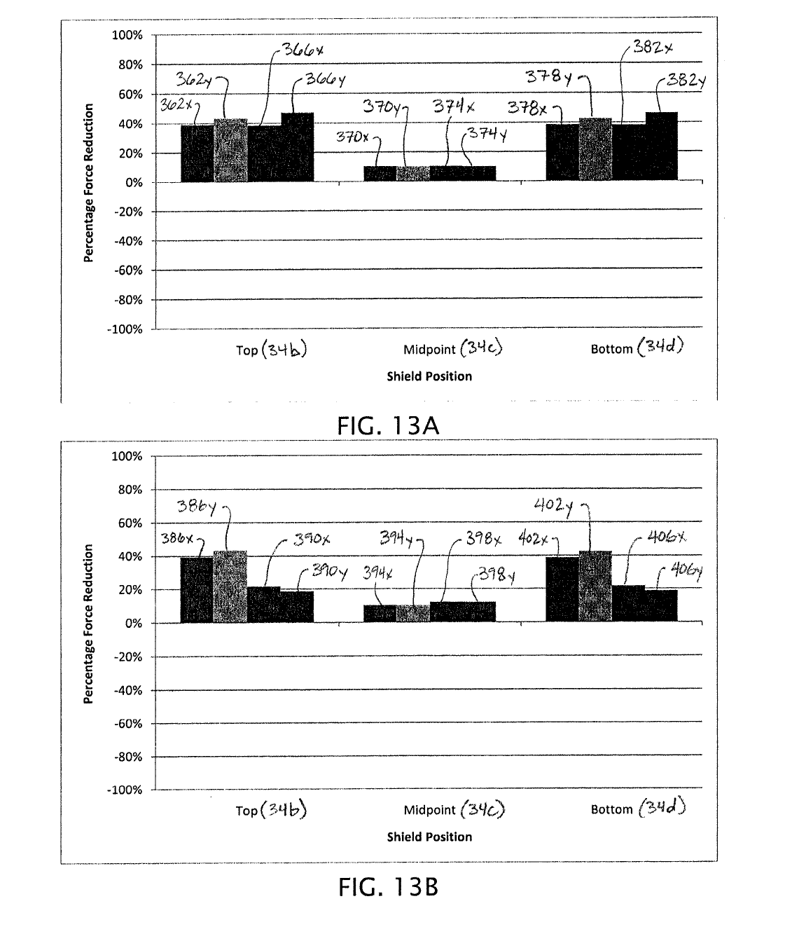

FIGS. 13A-13B depict the results of the simulations of FIGS. 11A-11B and 12A-12B. FIG. 13A depicts the reduction in force felt by each apparatus 34b, 34c, 34d in the X-direction and Y-direction for the apparatuses in which magnetically-permeable material 108 with the Shield-1 dimensions comprises either AISI 1010 steel or Carpenter 49 steel, relative to a similar apparatus without a bumper 104a. As such, a positive percentage force reduction in the chart corresponds to a reduction in force felt by the corresponding apparatus. Bars 362x and 362y correspond to the percentage reduction in force felt in the X and Y configurations of FIGS. 11A-11B and 12A-12B, respectively, by apparatus 34b in which magnetically-permeable material 108 comprises AISI 1010 steel. Bars 366x and 366y correspond to the percentage reduction in force felt in the X and Y configurations of FIGS. 11A-11B and 12A-12B, respectively, by apparatus 34b in which magnetically-permeable material 108 comprises Carpenter 49 steel. Bars 370x and 370y correspond to the percentage reduction in force felt in the X and Y configurations of FIGS. 11A-11B and 12A-12B, respectively, by apparatus 34c in which magnetically-permeable material 108 comprises AISI 1010 steel. Bars 374x and 374y correspond to the percentage reduction in force felt in the X and Y configurations of FIGS. 11A-11B and 12A-12B, respectively, by apparatus 34c in which magnetically-permeable material 108 comprises Carpenter 49 steel. Bars 378x and 378y correspond to the percentage reduction in force felt in the X and Y configurations of FIGS. 11A-11B and 12A-12B, respectively, by apparatus 34c in which magnetically-permeable material 108 comprises AISI 1010 steel. Bars 382x and 382y correspond to the percentage reduction in force felt in the X and Y configurations of FIGS. 11A-11B and 12A-12B, respectively, by apparatus 34c in which magnetically-permeable material 108 comprises Carpenter 49 steel.

FIG. 13B depicts the reduction in force felt by each apparatus 34b, 34c, 34d in the X- and Y-directions in which magnetically-permeable material 108 with either the Shield-1 or Shield-2 dimensions comprises AISI 1010 steel, relative to a similar apparatus without a bumper 104a. Bars 386x and 386y correspond to the percentage reduction in force felt by apparatus 34b in which magnetically-permeable material 108 has the Shield-1 dimensions. Bars 390x and 390y correspond to the percentage reduction in force felt by apparatus 34b in which magnetically-permeable material 108 has the Shield-2 dimensions. Bars 394x and 394y correspond to the percentage reduction in force felt by apparatus 34c in which magnetically-permeable material 108 has the Shield-1 dimensions. Bars 398x and 398y correspond to the percentage reduction in force felt by apparatus 34c in which magnetically-permeable material 108 has the Shield-2 dimensions. Bars 402x and 402y correspond to the percentage reduction in force felt by apparatus 34d in which magnetically-permeable material 108 has the Shield-1 dimensions. Bars 406x and 406y correspond to the percentage reduction in force felt by apparatus 34d in which magnetically-permeable material 108 has the Shield-2 dimensions.

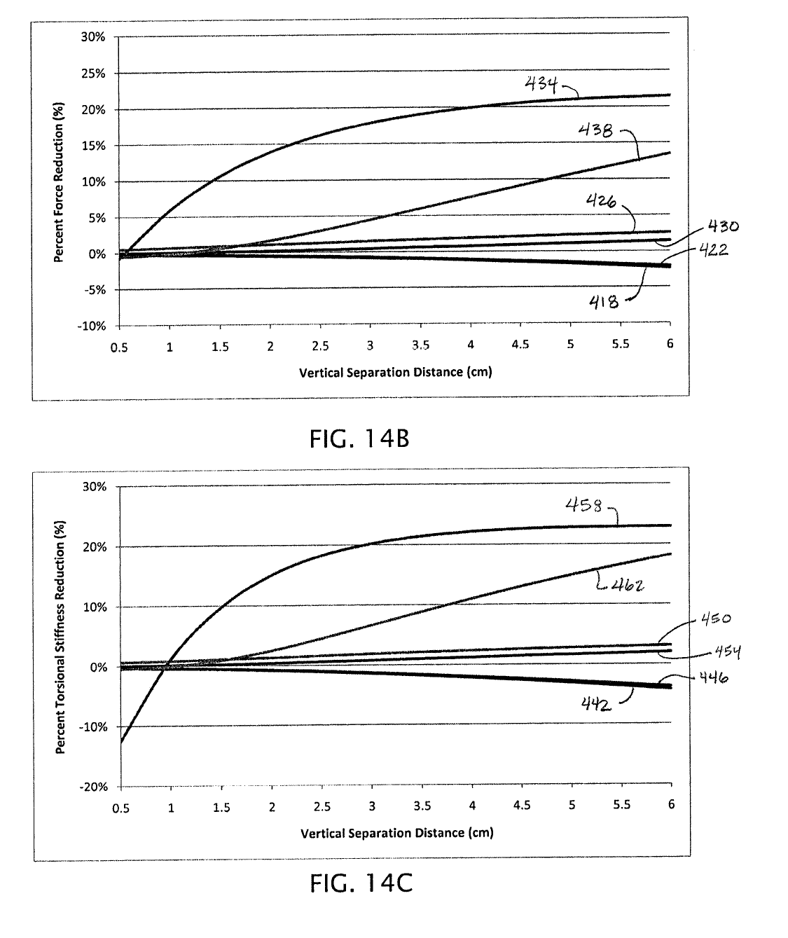

FIGS. 14A depicts perspective view of first and second elements 172 and 176 magnetically coupled to first and second elements 410 and 414 of a medical device (e.g., 38). First and second elements 410 and 414 can comprise at least one of a magnetically-attractive and a magnetically-chargeable material (e.g., a magnet, ferromagnetic material, paramagnetic material). For example, in the embodiment shown, first and second elements 410 and 414 each comprising a magnet, with one of elements 410 and 414 having an N-S magnetization and the other of elements 410 and 414 having an S-N magnetization. Likewise, in the embodiment shown, first and second elements 172 and 176 each comprise one or more magnets, with one of elements 172 and 176 having an N-S magnetization and the other of elements 172 and 176 having an S-N magnetization.

The simulation of FIG. 14A was performed for each of apparatuses 34b, 34c, and 34d, in which magnetically-permeable material 108 has either the dimensions of Shield-1 or Shield-2 and comprises AISI 1010 steel. FIG. 14B depicts the reduction in force felt by elements 410 and 414 at various values of distance 416 to elements 172 and 176, relative to force felt by elements 410 and 414 from a similar apparatus without a bumper 104a. As such, a positive percentage force reduction in the chart corresponds to a reduction in force felt by the corresponding apparatus. Curve 418 corresponds to the percentage reduction in force felt by elements 410 and 414 when magnetically coupled to apparatus 34b in which magnetically-permeable material 108 has the Shield-1 dimensions. Curve 422 corresponds to the percentage reduction in force felt by elements 410 and 414 when magnetically coupled to apparatus 34b in which magnetically-permeable material 108 has the Shield-2 dimensions. Curve 426 corresponds to the percentage reduction in force felt by elements 410 and 414 when magnetically coupled to apparatus 34c in which magnetically-permeable material 108 has the Shield-1 dimensions. Curve 430 corresponds to the percentage reduction in force felt by elements 410 and 414 when magnetically coupled to apparatus 34c in which magnetically-permeable material 108 has the Shield-2 dimensions. Curve 434 corresponds to the percentage reduction in force felt by elements 410 and 414 when magnetically coupled to apparatus 34d in which magnetically-permeable material 108 has the Shield-1 dimensions. Curve 438 corresponds to the percentage reduction in force felt by elements 410 and 414 when magnetically coupled to apparatus 34d in which magnetically-permeable material 108 has the Shield-2 dimensions.

FIG. 14C depicts the reduction in torque felt by elements 410 and 414 at various values of distance 416 to elements 172 and 176, relative to the torque felt by elements 410 and 414 from a similar apparatus without a bumper 104a. As such, a positive percentage reduction in the chart corresponds to a reduction in torque felt by the corresponding apparatus. Curve 442 corresponds to the percentage reduction in torque felt by elements 410 and 414 when magnetically coupled to apparatus 34b in which magnetically-permeable material 108 has the Shield-1 dimensions. Curve 446 corresponds to the percentage reduction in torque felt by elements 410 and 414 when magnetically coupled to apparatus 34b in which magnetically-permeable material 108 has the Shield-2 dimensions. Curve 450 corresponds to the percentage reduction in torque felt by elements 410 and 414 when magnetically coupled to apparatus 34c in which magnetically-permeable material 108 has the Shield-1 dimensions. Curve 454 corresponds to the percentage reduction in torque felt by elements 410 and 414 when magnetically coupled to apparatus 34c in which magnetically-permeable material 108 has the Shield-2 dimensions. Curve 458 corresponds to the percentage reduction in torque felt by elements 410 and 414 when magnetically coupled to apparatus 34d in which magnetically-permeable material 108 has the Shield-1 dimensions. Curve 462 corresponds to the percentage reduction in torque felt by elements 410 and 414 when magnetically coupled to apparatus 34d in which magnetically-permeable material 108 has the Shield-2 dimensions.

Embodiments of the present methods can include magnetically coupling one or more of the present apparatuses (e.g., 34, 34a, 34b, 34c, 34d) to a medical device (e.g., in a body cavity of a patient). For example, multiple ones of the present apparatuses (34, 34a, 34b, 34c, 34d) can be used in closer proximity to one another than otherwise feasible.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An apparatus comprising:
a platform configured to be magnetically coupled to a medical device disposed within a body cavity of a patient through a tissue, the platform comprising:
one or more elements comprising at least one of a magnetically-attractive material and a magnetically-chargeable material; and
a bumper extending around the one or more elements, the bumper comprising:
a magnetically-permeable material spaced apart from the one or more elements, the magnetically-permeable material configured to reduce the strength of a magnetic field of the one or more elements in at least one direction outside the bumper; and
a magnetically-inert material surrounding at least a portion of the magnetically permeable material.

2. The apparatus of claim 1, where in at least one point one the bumper, a cross-sectional area of the non-magnetic material is greater than a cross-sectional area of the magnetically-permeable material.

3. The apparatus of claim 2, where in a majority the bumper, the cross-sectional area of the non-magnetic material is greater than the cross-sectional area of the magnetically permeable bumper.

4. The apparatus of claim 1, where each of the one or more elements has a square cross-sectional shape.

5. The apparatus of claim 1, where each of the one or more elements comprises a magnet.

6. The apparatus of claim 1, where the bumper is spaced apart from an outer surface of the one or more elements by a substantially constant distance.

7. An apparatus comprising:
a platform configured to be magnetically coupled to a medical device disposed within a body cavity of a patient through a tissue, the platform comprising:
one or more elements comprising at least one of a magnetically-attractive material and a magnetically-chargeable material; and
a bumper extending around the one or more elements, the bumper comprising:
a magnetically-permeable material spaced apart from the one or more elements, the magnetically-permeable material configured to reduce the strength of a magnetic field of the one or more elements in at least one direction outside the bumper.

8. The apparatus of claim 7, where the bumper further comprises a magnetically-inert material surrounding at least a portion of the magnetically-permeable material.

9. The apparatus of claim 7, where each of the one or more elements has a square cross-sectional shape.

10. The apparatus of claim 7, where each of the one or more elements comprises a magnet.

11. The apparatus of claim 7, where the bumper is spaced apart from an outer surface of the one or more elements by a substantially constant distance.

12. An apparatus comprising:
a platform configured to be magnetically coupled to a medical device disposed within a body cavity of a patient through a tissue, the platform comprising:
two or more elements each comprising at least one of a magnetically-attractive material and a magnetically-chargeable material; and
a bumper extending around the two or more elements, the bumper comprising:
a magnetically-permeable material spaced apart from the two or more elements, the magnetically-permeable material configured to reduce the strength of a magnetic field of the one or more elements in at least one direction outside the bumper.

13. The apparatus of claim 12, where the bumper further comprises a magnetically-inert material surrounding at least a portion of the magnetically-permeable material.

14. The apparatus of claim 12, where each of the two or more elements has a square cross-sectional shape.

15. The apparatus of claim 12, where each of the two or more elements comprises a magnet.

16. The apparatus of claim 15, where the two or more elements comprises two elements having substantially opposite magnetic orientations.

17. The apparatus of claim 12, where the bumper is spaced apart from an outer surface of the two or more elements by a substantially constant distance.

18. The apparatus of claim 1, where the magnetically permeable material of the bumper has a substantially constant cross-sectional shape.

19. The apparatus of claim 18, where the cross-sectional shape is substantially rectangular.

20. The apparatus of claim 1, where the apparatus is disposed outside a body cavity of a patient and is magnetically coupled to a medical device within the body cavity.
